(19) United States
(12) Patent Application Publication
(10) Pub. No.: US 2003/0084909 A1
(43) Pub. Date: May 8, 2003

(54) Apparatus and method for three dimensional spatial registration of surgical procedures using radio tagging

(76) Inventors: Everette C. Burdette, Champaign, IL (US); Dana L. Deardorff, Oakland, CA (US)

Correspondence Address: FOLEY & LARDNER, 330 NORTH WABASH AVENUE, SUITE 3300, CHICAGO, IL, 60611-3608, US

(21) Appl. No.: 10/003,612

(22) Filed: Nov. 2, 2001

(51) Int. Cl. A61N 5/00; A61B 17/00; A61F 7/12; A61B 19/00

(52) U.S. Cl. 128/899; 606/1; 600/1; 607/113

(57) ABSTRACT

An apparatus for the three-dimensional spatial registration of components of a surgical procedure. At least one radio tag is incorporated into a medical device. The at least one radio tag provides a radio signal source to a radio receiver for determining the position of the signal source. A central control unit includes software controls for providing information concerning the position of the signal source, and data is communicated between the central control unit and radio receiver. The central control unit includes a graphical interface for viewing the position of the radio signal sources.

### FIELD OF THE INVENTION

The present invention relates generally to the spatial registration of treatment procedures. More particularly, the present invention relates to the design and use of an integrated system for three-dimensional spatial registration of minimally invasive treatment procedures.

### BACKGROUND OF THE INVENTION

Minimally invasive devices and surgical procedures have the potential to provide patients with a number of clinical benefits by significantly reducing the amount and level of pain, surgical complications, morbidity, blood loss, treatment time, and treatment cost. However, a significant limitation for many minimally invasive procedures is the lack of real-time image guidance or spatial registration of the therapy device, the treatment process and the patient treatment portion. Without real-time image guidance or spatial registration of the therapy device and treatment process with the portion of the patient being treated, the procedure is performed “blindly” by the clinician, and evaluation of the treatment process is typically not available until post-treatment imaging techniques such as magnetic resonance imaging, computed tomography (CT) or ultrasound are utilized.

One example of this significant limitation is in the current procedures of minimally invasive brachytherapy radiation treatment. Brachytherapy radiation is commonly used as interstitial implants for the treatment of prostate cancer, and is also used with intravascular delivery to prevent restenosis of coronary artery disease. During these procedures, imaging techniques such as ultrasound are used to spatially register and visualize the local anatomy. The exact placement of the radiation seeds in the tissue, however, is not precisely known. This is due primarily to difficulties with visualizing individual radiation sources, as well as with spatially registering the position of these sources within the three-dimensional treatment. Placement issues also arise in the case when radioactive line sources or sources of any other type of ionizing radiation are used. Because the spatial registration of the radiation is not precisely known, exact dosimetry in real-time is not possible, and separate post-treatment imaging, typically multiple CT slices, must be used to validate the position of the implanted seeds. Similar issues relating to placement localization also arise when RF, acoustic, and laser ablative localization and anatomical or treatment region registration.

Additionally, there are other situations where the precise position of various therapeutic and diagnostic devices would aid a user. For example, the position of RF sources, acoustic source, and laser abalation sources is often not precisely known. It would also be beneficial to know the exact position of a surgical instrument during placement inside a body, as well as the position of other anatomical features relative to the placed radioactive seeds and other sources.

A variety of types of radio tags are conventionally known for registering the position of an item to which a tag is attached, but some types suffer from a number of drawbacks. One such type of radio tag system 15 which could not be utilized with the present invention has been developed by Motorola Corporation and is shown generally in FIG. 3. A computer chip 40 is operatively connected to a supply of conductive carbon ink 42. In one particular preferred embodiment, the computer chip 40 is formed from silicon. When the computer chip 40 and the conductive carbon ink 42 are placed within range of a reader 44, the conductive carbon ink 42 picks up electrostatic charges from the reader 44. These electrostatic charges create a current across the computer chip 40, providing sufficient power to the computer chip 40. The reader 44 then proceeds to transmit information from the computer chip 40 to a central processing unit 46, such as a computer. Alternatively, a metal coil, shown in phantom at 47, can be used instead of the conductive carbon ink 42 in order to provide a sufficient current across the computer chip 40. Other conventional methods of inductive or capacitive coupling can also be used. The radio tag 15 includes a transmitter that emits an electromagnetic signal in the radiofrequency range or a higher range, such as the microwave range. Radio tags 15 of this type, however, are relatively large in size, making their use impractical on small surgical devices or implants. These types of radio tags also have a limited range in which their signal can be received. Furthermore, this type of radio tag system does not register multiple points in space at one time. Additionally, many radio tags are of a size of approximately 1 centimeter in length, which is very large when used for implantation purposes.

Another limitation of conventionally known radio tagging systems relates to the ability to localizing individual signals. Conventionally known systems do not provide for any type of guidance, localization or registration of the individual tags. Essentially, the sensor or receiver that is used in such systems comprise means for detecting a signal within a certain distance. There is no stereotactic frame, however, that localizes the precise location of the tags.

### SUMMARY OF THE INVENTION

It is therefore an object of the invention to provide an improved treatment device and technique that allows substantially exact spatial registration during the procedure of placing a probe, needle, or identification marker inside or on a patient's body.

It is another object of the invention to provide an improved treatment device and technique that allows substantially exact spatial registration during the placement of individual radiation sources or seeds inside or on the patient's body.

It is yet another object of the invention to provide an improved relatively simple and cost-effective system for spatial registration using radio tagging for 3-D spatial registration of surgical procedures.

Further advantages and features of the present invention will be apparent from the following specifications and claims illustrating the preferred embodiments of the present invention.

### BRIEF DESCRIPTION OF THE DRAWINGS

FIG. 1 is a perspective view of a minimally invasive surgical device and related equipment using three dimensional surgical radio-tagging during a surgical procedure according to one embodiment of the present invention;

FIG. 2 is a representation of a graphical display of the treatment region during the surgical procedure; and

FIG. 3 is a representation of the operation of a radio tag using conductive carbon ink to generate a current between the radio tag and a reader or receiver.

### DETAILED DESCRIPTION OF THE INVENTION

The present invention relates to the design and use of a system for three-dimensional spatial registration of minimally invasive treatment procedures. The present invention is primarily intended for the application of brachytherapy for prostate cancer and to prevent restenosis of CAD, although the invention can be used for a variety of therapy procedures which can be implemented with the subject system.

One preferred embodiment of the invention includes an apparatus with an integrated system of both hardware and software. The primary elements comprise at least one radio signal tag, a reader system for receiving the positional radio signal from the at least one radio signal tag, and a central control system in the form of a computer with integrated software to receive and process positional data. The radio signal tag serves as a transmitter. The integrated software is responsible for internal control and display of all primary functions of the central control system as well as all data calculations and manipulations. Importantly, the control system and reader do not interfere with existing diagnostic imaging systems, such as ultrasound imaging systems, for spatial registration with these images.

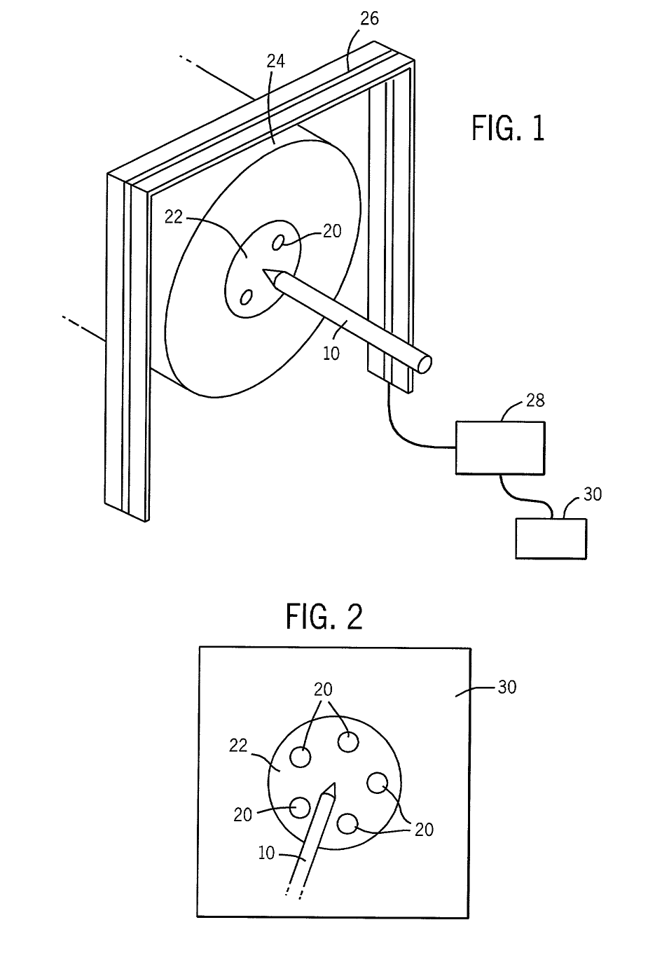

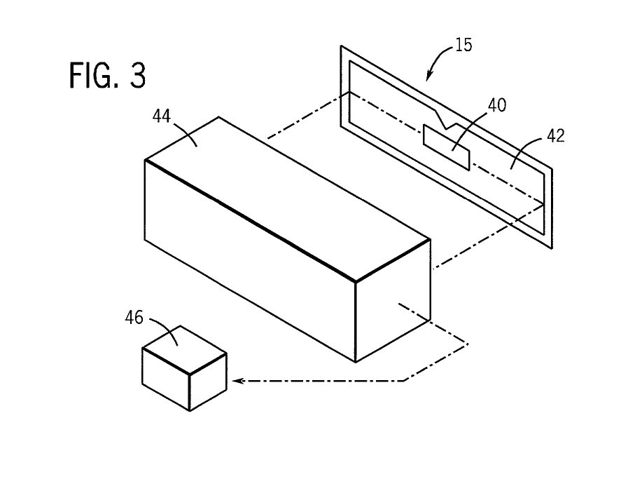

FIGS. 1 and 2 show the general layout and operation of a surgical procedure using radio tagging according to one embodiment of the invention. FIG. 1 shows a minimally invasive surgical device 10 and a plurality of the radio tags 20 placed inside a treatment region 22 of a patient's body 24. The radio tags 20 are substantially smaller than the 1 centimeter tags that are conventionally used, but could be based upon the same technology while using similar technology. Surrounding the treatment region 22 is a detector array or receiver 26 for spatial localizing signals from each of the plurality of radio tags 20. The detector array 26 may also serve as the signal reader or receiver and is operatively connected to a central processing unit 28 such as a personal computer. The detector array 26 may also serve as a coordinated spatial reference frame for the imaging of the individual radio tags 20 and the treatment region 22. In one particular embodiment of the invention, three or more separate receivers are used in order to triangulate the position of the individual radio tags 20.

As shown in FIGS. 1 and 2, the central processing unit 28 incorporates the signals from the radio tags 20 into a graphical display 30 that shows the radio tag positions and images of the surrounding anatomical region. In one preferred embodiment of the invention, the graphical display 30 shows a three-dimensional volume rendering of the radio tag positions and anatomical features. It is also possible, however, for the graphical display 30 to show a two-dimensional rendering of the radio tag positions and anatomical features. The graphical display 30 spatially registers the radio tags 20 to an external coordinate system or reference frame. The external coordinate system or reference frame may consist of a plurality of diagnostic image slices or volumes. Depending upon the particular needs and system specifications, the registration of the signals may appear in either two dimensions or three dimensions.

In one particular embodiment of the invention, each sensor of the radio tags 20 is integrated with a particular radiation seed, and the radiation seeds are used during brachytherapy procedures. Alternatively, the radio tags 20 could also be placed on devices such as probes, markers, needles, catheters or other instruments for spatial localization. The radio tags 20 could therefore be used for both therapeutic and diagnostic purposes. Because a number of signals of different types can be used simultaneously, in one embodiment of the invention each radio signal is unique in frequency, pulse length or modulation and power in order to differentiate it from signals of other ones of the radio tags 20 or other sources of signals.

The present invention includes a number of substantial benefits which cannot otherwise be achieved using conventionally known methods. For example, the present invention provides for the transmission and reception of the radio tag signal, allowing for accurate positional data of a variety of types of surgical components. The use of the radio tag signal also allows for the real-time calculation of the position of a moving signal source. The radio tag position can also be spatially registered in substantially real time in relation to an external coordinate system, such as anatomicl features of a patient's body and/or images from an imaging system. The source position can also be displayed in registration with the external coordinate system. In one embodiment of the invention, the source position can be graphically displayed in substantially real time with approximately less than a second of delay.

The device and method of the present invention allows a clinician to view the position of the signal source in real time, in three-dimensional space, and in spatial registration within a coordinate system reference framework or images. There are several different physical embodiments of the apparatus to accomplish the spatial registration, and the present disclosure is not intended to limit these possible design variations.

While the preferred embodiments of the invention have been described, it will be understood by those skilled in the art to which the invention pertains that numerous modifications and changes may be made without departing from the true spirit and scope of the invention. It is accordingly intended to define the scope of the invention precisely in the claims appended to and forming a part of this application.

1. An apparatus for the three-dimensional spatial registration of surgical procedures, comprising:
at least one radio tag incorporated into a medical device, the at least one radio tag providing a radio signal source;
a radio receiver for determining the position of the signal source; and
a central control unit including software controls for providing information concerning the position of the signal source.

2. The apparatus of claim 1, further comprising a communication couple between the central control unit and radio receiver.

3. The apparatus of claim 2, wherein the central control unit includes user interface input and graphic display output capabilities.

4. The apparatus of claim 3 wherein the at least one radio tag comprises a transmitter that emits an electromagnetic signal in the radiofrequency range or higher frequency.

5. The apparatus of claim 4 wherein the transmitter is powered by at least one of an inductive coupling and a capacitive coupling from an external field source.

6. The apparatus of claim 5, wherein the at least one radio tag is integrated with at least one of a radiation source used for brachytherapy and a surgical device.

7. The apparatus of claim 5, wherein the at least one radio tag is integrated with a thermotherapy device.

8. The apparatus of claim 5, wherein the signal from each of the at least one radio tag is unique to differentiate each signal from other source signals used simultaneously.

9. The apparatus of claim 1, comprising at least two separate radio receivers for establishing the positions of the at least one radio tag signal.

10. The apparatus of claim 9, comprising at least three separate radio receivers for triangulating the positions of the at least one radio tag signal.

11. The apparatus of claim 8, wherein the central control unit determines and displays the spatial position of each source signal in substantially real time.

12. The apparatus of claim 1, wherein the central control unit and the radio receiver are spatially registered with at least one of an external coordinate system, a reference frame and a localization reference device.

13. The apparatus of claim 12, wherein the external coordinate system or reference frame is based on a diagnostic image or image volume.

14. The apparatus of claim 5, wherein the radio signal is digitally encoded.

15. A method for the three-dimensional spatial registration of a minimally invasive surgical procedure, comprising the steps of:
placing a device including a radio tag into or on a patient's body, the radio tag emitting a radio signal source;
using a radio receiver to determine the position of the radio signal source; graphically displaying the position of the radio signal source.

16. The method of claim 15, wherein the radio tag is incorporated into or on at least one of a thermotherapy device, a surgical abalation device and a surgical device.

17. The method of claim 15, wherein the radio tag is incorporated into or on one of a radiation source or seed for brachytherapy.

18. The method of claim 15, wherein the position of the radio signal source is spatially registered to an external coordinate system or reference frame, and wherein the position of the radio signal source graphically is displayed in relation to the coordinate system or reference frame.

19. The method of claim 17, wherein the external coordinate system comprises a plurality of diagnostic image slices or volumes.

20. The method of claim 15, wherein the radio signal source is graphically displayed in substantially real time.

21. The method of claim 15, wherein the external coordinate system comprises a single diagnostic image slice.

22. The method of claim 15, wherein the external coordinate system comprises a single diagnostic image volume.
